SEQUENCE LISTING (14 sequences)

SEQ ID NO: 1 - DNA, 21 bp, Artificial starter
  B2M Forward Starter sequence
tcaggtttactcacgtcatcc

SEQ ID NO: 2 - DNA, 20 bp, Artificial
  B2M Reverse Starter sequence
acacggcaggcatactcatc

SEQ ID NO: 3 - DNA, 20 bp, Artificial
  GATA-4 Forward Starter sequence
aaacggaagcccaagaacct

SEQ ID NO: 4 - DNA, 20 bp, Artificial
  GATA-4 Reverse Starter sequence
actgagaacgtctgggacac

SEQ ID NO: 5 - DNA, 20 bp, Artificial
  NKX2-5 Forward Starter sequence
tgtccacgctgcatggtatc

SEQ ID NO: 6 - DNA, 20 bp, Artificial
  NKX2-5 Reverse Starter sequence
gatcactcattgcacgctgc

SEQ ID NO: 7 - DNA, 21 bp, Artificial
  MEF2C Forward Starter sequence
ccaacttcgagatgccagtct

SEQ ID NO: 8 - DNA, 21 bp, Artificial
  MEF2C Reverse Starter sequence
gtcgatgtgttacaccaggag

SEQ ID NO: 9 - DNA, 22 bp, Artificial
  MYH7B Forward Starter sequence
gcaataaaaggggtagcagagc

SEQ ID NO: 10 - DNA, 22 bp, Artificial
  MYH7B Reverse Starter sequence
gactccccaagttcactcacat

SEQ ID NO: 11 - DNA, 20 bp, Artificial
  TNNT2 Forward Starter sequence
ctggccattgaccacctgaa

SEQ ID NO: 12 - DNA, 20 bp, Artificial
  TNNT2 Reverse Starter sequence
gctgcttgaacttctcctgc

SEQ ID NO: 13 - DNA, 20 bp, Artificial
  GJA1 Forward Starter sequence
tctctcatgtgcgcttctgg

SEQ ID NO: 14 - DNA, 20 bp, Artificial
  GJA1 Reverse Starter sequence
tgacaccatcagtttgggca

(19) United States
(12) Patent Application Publication
(10) Pub. No.: US 2016/0193250 A1
(43) Pub. Date: Jul. 7, 2016

(54) CHORION-DERIVED MSCS CELLS AND CONDITIONED MEDIA AS INDUCER FOR ANGIOGENESIS APPLICATION FOR THE TREATMENT OF CARDIAC DEGENERATION.

(71) Applicant: CELLS FOR CELLS, Santiago (CL)

(72) Inventors: Maroun Khoury, Santiago (CL); Paz Gonzalez Lorca, Santiago (CL)

(21) Appl. No.: 14/894,974

(22) PCT Filed: May 30, 2014
(86) PCT No.: PCT/IB14/61859

§ 371 Date: Nov. 30, 2015

Related U.S. Application Data
(60) Provisional application No. 61/828,879, filed on May 30, 2013.

(51) Int. Cl. A61K 35/28; C12N 5/073

(52) U.S. Cl. CPC A61K 35/28; C12N 5/0605

(57) ABSTRACT

The present invention relates to a method for providing chorion cells as a source of MSCs with cardio-myogenic and angiogenic potential, the use of these cells in clinical treatment, the obtaining of a conditioned medium of chorion-MSCs cells as inducer of angiogenesis and its use in tube-like structures generation and cardiac regeneration as an alternative to bone marrow cells in the treatment of degenerative conditions.

### BACKGROUND OF THE INVENTION

1. Technical Field

The present invention relates to a method for providing chorion cells as a source of MSCs with cardio-myogenic and angiogenic potential, the use of these cells in clinical treatment, the obtaining of a conditioned medium of chorion-MSCs cells as inducer of angiogenesis and its use in tube-like structures generation and cardiac regeneration as an alternative to bone marrow cells in the treatment of degenerative conditions.

2. Background Art

Due to the pre-clinical and clinical promissory results, therapy with mesenchymal stem cells (MSCs) has become a real strategy to treat different degenerative diseases. The most used source is bone marrow; however, there are different limitations to this type of cells, such as the invasive and painful method to obtain them, complications with the donors' age, and the obtaining of a small quantity of primary tissue.

There are no such limitations when using human delivered placenta as cell source.

Cardiac insufficiency is identified as one of the main causes of death in the entire world, and currently available therapies could improve patients' prognosis, but there are no treatments focusing on regenerating damaged cardiac tissue. Cell therapy has become an alternative therapeutic intervention for the treatment of cardiovascular diseases, and many researchers have focused on the development of therapies with adult stem cells such as MSCs.

Bone marrow is the common source to obtain MSCs, but it has a series of inconvenients, being a highly invasive way to obtain them through a biopsy, and its number of initial MSCs is very low. Several research groups have indicated human placenta as an alternative source of MSCs because this tissue is able to eliminate risk for the donor, being a tissue discarded after birth.

Angiogenesis or revascularization is the process to generate new blood vessels derived as extensions of the existing vasculature. Cells mainly implied in this process are endothelial cells, which line all blood vessels and are practically the total capillary.

### Description of Placenta Tissues as Potential Source of Cell Therapy

Human placenta has an essential role in fetal development by providing nutrition and supporting immune tolerance. Placenta is formed by two main fetal structures: shaggy chorion (also known as chorionic plate), mainly in charge of placenta blood circulation and the amniotic sac, whose main function is creating the amniotic cavity that acts as the embryo physical protection. In close association with both structures, there is the mother part of the placenta called decidua, a tissue that covers the gestating uterus and will form the mother-fetus interface.

From the shaggy chorion, the oxygenated and nutrient-rich blood goes to the embryo through the umbilical cord, which is anatomically considered part of fetal membranes. The umbilical cord has two arteries and one vein rolled in the form of a spiral, immersed in a transparent gelatin called gelatin of Wharton.

MSCs Derived from Placenta

MSCs have been obtained from different structures forming the placenta, studies have established that they meet the International Society for Cell Therapy, ISCT, requirements, and it has been reported that they express a variety of totipotence markers such as OCT-4, SOX-2, SSEA-1, and SSEA-3, in addition to confirming that it resists culture conditions in hypoxia and serum deprivation. Although there are different researches that assess phenotypic, proliferation, and differentiation characteristics of MSCs isolated from chorion, decidua, and gelatin of Wharton, there is no comparative work on these areas regarding a therapeutic potential, particularly in vitro.

The majority of clinical trials have been carried out using adipose tissue, BM-MSCs, and MSCs. Table 1 shows the on-going clinical trials using MSCs derived from some area of placenta. It is important to note that the majority of studies are using mesenchymal stem cells from MSCs from umbilical cord (UC-MSCs) and, when they refer to placenta mesenchymal stem cells (PL-MSCs), they do not distinguish whether they are using MSCs derived from chorion (CHOR-MSCs) or deciduas (DEC-MSCs), or a mixture of both. There are no clinical trials using MSCs derived from placenta to treat heart conditions, such as cardiac insufficiency, IC, (www.clinicaltrials.gov, ClinicalTrials.gov. US National Institutes of Health. National Library of Medicine. Reviewed in May 2013).

Due to these records, it is very important to establish whether the MSCs obtained from different areas forming the placenta present differences regarding therapeutic potential for cardiac diseases. This becomes much more important if we consider that, due to ISCT standards, it is not possible to carry out therapy with mixed cells from two individuals in the event the placenta is of fetal and maternal origin, which would carry important regulation implications in the event current trials have not determined maternal and fetal cells contamination in their samples.

Chorion, decidua, and umbilical cord are components of human placenta representing different sources of MSCs, which provide significant advantages regarding MSCs from bone marrow, such as: reduced invasion, they correspond to young tissue and there is a larger amount of primary tissue at the extraction. They are of fetal origin (chorion and umbilical cord) or maternal (decidua) and have different physiological functions, which determine possible differences of characteristics such as differentiation and secretion potential of useful factors for positive effect in decompensate biologic systems. According to the observations above, this study sets forth that MSCs isolated from chorion, umbilical cord, and decidua present differences regarding in vitro cardio-myogenic and angiogenic potential. Also, the MSCs placenta source outstanding above the rest of sources shows an in vitro cardio-myogenic and angiogenic potential equal or higher than the MSCs derived from bone marrow.

TABLE 1
Current Clinical Trials appearing in www.clinicaltrials.gov
(research carried out in May 2013) Using MSCs Derived from Placenta.

| Type of MSC | | | |
|---|---|---|---|
| Identification N^{o} | Disease | Stage | Description |
| UCB-MSCs | Type 2 diabetes | 1 and 2 | Safety and Efficacy Study of Umbilical |
| NCT01413035 | | | Cord/Placenta-Derived Mesenchymal Stem |
| | | | Cells to Treat Type 2 Diabetes. Patients being |
| | | | recruited. 2011-2014. |
| UC-MSCs | Severe aplastic | 2 | Safety and Efficacy Study of Umbilical |
| NCT01182662 | anemia | | Cord/Placenta-Derived Mesenchymal Stem |
| | | | Cells to Treat Severe Aplastic Anemia. Patients |
| | | | being recruited. 2010-2013. |
| UC-MSCs | Ankylosing | 1 | Safety and Efficacy Study of Umbilical |
| NCT01420432 | spondilytis | | Cord/Placenta-Derived Mesenchymal Stem |
| | | | Cells to Treat Ankylosing Spondylitis (AS). |
| | | | Patients being recruited. 2011-2013. |
| UC-MSCs | Myelodysplastic | 2 | Safety and Efficacy Study of Umbilical |
| NCT01129739 | syndromes | | Cord/Placenta-Derived Mesenchymal Stem |
| | | | Cells to Treat Myelodysplastic Syndromes. |
| | | | Patients being recruited. 2010-2013 |
| PL-MSCs | Idiopathic | 1 | A Study to Evaluate the Potential Role |
| NCT01385644 | pulmonary | | of Mesenchymal Stem Cells in the Treatment of |
| | fibrosis | | Idiopathic Pulmonary Fibrosis (MSC in IPF). On- |
| | | | going, with no patient recruiting. 2010-2013. |
| UC-MSCs | Graft-versus- | 1 y 2 | Allogeneic Mesenchymal Stem Cell for Graft- |
| NCT00749164 | host disease | | Versus-Host Disease Treatment (MSCGVHD). |
| | | | Has not been updated. 2009-2012. |
| UC-MSCs | Rheumatoid | 1 y 2 | Safety and Efficacy Study of Umbilical Cord- |
| NCT01547091 | arthritis | | Derived Mesenchymal Stem Cells for |
| | | | Rheumatoid Arthritis (RA). Not patients being |
| | | | recruited. 2012-2013 |
| UC-MSCs | Liver failure | 1 y 2 | Umbilical Cord Mesenchymal Stem Cells |
| NCT01724398 | | | Transplantation Combined With Plasma |
| | | | Exchange for Patients With Liver Failure. |
| | | | Patients being recruited. 2012-2015 |
| UC-MSCs | Liver failure | 1 y 2 | Safety and Efficacy of Human Mesenchymal |
| NCT01218464 | | | Stem Cells for Treatment of Liver Failure. |
| | | | Patients being recruited. 2009-2014 |
| UC-MSC | Alzheimer | 1 y 2 | Safety and Efficiency of Umbilical Cord-derived |
| NCT01547689 | disease | | Mesenchymal Stem Cells (UC-MSC) in Patients |
| | | | With Alzheimer's Disease (SEMAD). No patients |
| | | | being recruited. 2012-2014. |

UCB-MSCs: Mesenchymal stem cells from umbilical cord blood
UC-MSCs: Mesenchymal stem cells from umbilical cord (gelatin of Wharton)
PL-MSCs: Mesenchymal stem cells from placenta (whether chorion or decidua not specified)

In the prior art, document WO2008146992 provides a method for isolating mesenchymal stem cells derived from a placental chorionic plate membrane, the method including: (a) harvesting a chorionic plate membrane from a detached placenta; (b) harvesting cells present in the chorionic plate membrane obtained in step (a) by scraping; (c) adding a solution containing trypsin and ethylenediaminetetraacetate to the cells obtained in step (b) to perform an enzymatic reaction and adding a fetal bovine serum thereto to terminate the enzymatic reaction; and (d) centrifuging the reaction solution obtained in step (c) and culturing the obtained cells in a medium containing a fetal bovine serum and an antibiotic. Mesenchymal stem cells isolated according to this method are used as human feeder cells for culturing human embryonic stem cells, but it is not shown any evidence of advantages of this cells compared to other types of placental tissue or evidence of the application of the mesenchymal stem cells isolated according to this method as differentiated cells for the treatment of degenerative diseases.

Usually, the placental tissue obtained comprises amnion, chorion and decidua, as shown in document EP1845154 where placenta tissue-derived multipotent stem cells and cell therapeutic agents containing the same are obtained. The method comprising culturing amnion, chorion, decidua or placenta tissue in a medium containing collagenase and bFGF and collecting the cultured cells showing a positive immunological response to CD29, CD44, CD73, CD90 and CD105, and showing a negative immunological response to CD31, CD34, CD45 and HLA-DR; showing a positive immunological response to Oct4 and SSEA4; growing attached to plastic, showing a round-shaped or spindle-shaped morphology, and forming spheres in an SFM medium so as to be able to be maintained in an undifferentiated state for a long period of time; and having the ability to differentiate into mesoderm-, endoderm- and ectoderm-derived cells.

### BRIEF DESCRIPTION OF THE DRAWINGS

FIG. 1: Fibroblastoide morphology of placenta and bone marrow MSCs during in vitro expansion for cardio-myogenesis and vasculogenesis experiments. Mesenchymal stem cells (MSCs) from (A) bone marrow (BM-MSCs), (B) umbilical cord (UC-MSCs), (C) chorion (CHOR-MSCs), and (D) decidua (DEC-MSCs) are shown. Images were obtained with 10× magnifying.

FIG. 2: Placenta and bone marrow MSC morphology during cardio-myogenic induction. (A) 15 days culture after treatment with 5-azacytidine (5-aza) 10 μM for 24 hours. Images representing placenta and bone marrow MSC morphology in normal culture conditions (upper) and after treatment with 5-aza (lower). Images were obtained with 1× magnifying. Images representing (B) the presence of vacuoles in MSCs treated with 5-aza. (C) Circular MSCs, (D) tubular MSCs; arrows indicate connections (20× magnifying).

FIG. 3. Scheme showing the expression of genes involved in cardio-myogenesis. Transcription factors NKX2-5, GATA-4, and MEF2C increase their expression during the first differentiation stages, while non-differentiation markers such as OCT-4 and SOX-2 decrease their expression as the cell specializes in a myocardial phenotype. Action of transcription factors (blue) activate the codifying genes expression for structural cardiac proteins such as MYH7B (heavy chain myosin), GJA1 (conexine-43), and TNNT2 (cardiac troponin).

FIG. 4. RT-qPCR efficiency curves for B2M standardizing gene and for genes involved in cardiac differentiation: NKX2-5, GATA-4, MEF2C, MYH7B, GJA1, TNNT2. Curves were carried out with cDNA serial dilutions (1:5 to 1:10000). Efficiency percentage was calculated using the slope of the line, as specified in Table 2.

FIG. 5: 5-azacitidina (5-aza) induces the expression of genes involved in cardio-myogenesis in MSCs. The genes expression (A) NKX2-5, (B) GATA-4, (C) MEF2C, (D) MYH7B, (E) GJA1, and (F) TNNT2 was detected through RT-qPCR in MSCs derived from bone marrow (BM), umbilical cord (UC), chorion (CHOR), and decidua (DEC), treated during 24 h with 10 μM 5-AZA and cultured for 30 days after removing 5-aza. ***p<0.001 compared to the control group (n=3). ### p<0.001, CHOR-MSCs compared to DEC-MSCs and UC-MSCs.

FIG. 6: Cardiac troponin expression in MSCs treated with 5-aza 10 μM for 24 h and cultured during 30 days after removing the treatment. Images are representative of indirect immunofluorescence from (A) BM-MSC, (B) UC-MSC, (C) CHOR-MSC, and (D) DEC-MSC. (E) Secondary antibody control. Quantification of positive cells number. *P<0.05 BM-MSCs with regard to CHOR-MSCs and DEC-MSCs (N=3). Images obtained with immersion objective at 40× magnifying.

FIG. 7: Conexine 43 expression in MSCs treated with 5-aza 10 μM for 24 h and cultured during 30 days after removing treatment. Images representing indirect immunodeficiency in (A) BM-MSC, (B) UC-MSC, (C) CHOR-MSC, and (D) DEC-MSC, (E) Secondary antibody control, (F) Quantification of positive cells number *p<0.05, **p<0.01, ***p<0.001 CHOR-MSCs with regard to BM-MSCs, UC-MSCs, and DEC-MSCs (n=3). Images obtained through immersion objective at 40× magnifying.

FIG. 8: In vitro forming of tube-like structures by MSCs through the matrigel assay. (A) Images representative of MSCs on matrigel cultured with media for endothelial cells (EGM) for 5 h. A quantitative analysis is shown, comparing different cell sources to (B) Total ramification points, (C) Total formed loops, (D) Total tube length. ***P<0.001 CHOR-MSC with regard to UC-MSC, DEC-MSC, and BM-MSC, (n=3). ### p<0.001, ## p<0.01 MSC-CHOR with regard to human umbilical vein endothelial cell, HUVEC, (n=3). Images obtained with 4× magnifying.

FIG. 9: MSC supernatants under hypoxia and normoxia conditions induce the forming of in vitro tube-like structures through assay with matrigel reduced to factors. Representative images showing (A) Negative control using DMEM and positive control using EGM, (B) Induction of the bone marrow mesenchymal stem cell (sBM-MSC), umbilical cord (sUC-MSC), chorion (sCHOR-MSC), and decidua (sDEC-MSC) supernatant in vitro forming obtained under hypoxia and normoxia conditions. Angiogenic potential quantitative analysis regarding (C) Total ramification points, (D) Total loops formed, (E) Tubes total length. ***p 0.001 sCHOR-MSC with regard to other MSC sources under hypoxia or normoxia conditions. ++p 0.01 sCHOR-MSC with regard to EGM and ### p<0.001, ## p<0.01 and # p<0.05 MSCs in hypoxia with regard to normoxia. Images taken with 4× magnifying.

FIG. 10. Quantification of soluble factors release involved in the angiogenesis in the supernatant of MSCs under hypoxia and normoxia condition.

FIG. 11. Representative images of the differences between the in vitro tube formation EGM and S-CHOR.

FIG. 12. Doubling time of MSCs obtained from placenta and bone marrow.

FIG. 13. Isotype control, control without secondary antibody, and positive control.

FIG. 14. Calculation of classic marker profile for MSCs characterization.

FIG. 15. Percentage of positive population for placenta and bone marrow for a MSCs marker classic profile.

FIG. 16. Calculation of additional markers profile in MSCs.

FIG. 17. Percentage of positive population for additional cell markers.

FIG. 18. Calculation of embryo markers in placenta and bone marrow MSCs.

FIG. 19. Percentage of positive cells for embryo markers in placenta and bone marrow MSCs.

FIG. 20. Assessment of the differentiation capacity of MSCs derived from placenta and bone marrow to adipocyte, chondrocyte, and osteocyte.

### BRIEF DESCRIPTION OF THE INVENTION

The present invention consists of a method for providing chorion cells as a source of MSCs with cardio-myogenic and angiogenic potential and the use of these cells in clinical treatment used in tube-like structures generation and cardiac regeneration as an alternative to bone marrow cells in the treatment of degenerative conditions.

Furthermore, in vitro conditioned media can be used as source of growth factors for the pre-treatment and culture of different types of cells for the use in vivo in cell growth and regeneration with applications in degenerative diseases as raw material in products for muscle regeneration for high-performance sportspersons (Quintero A, Clin Sports Med. 2009) or cosmetic products, among other commercial applications resulting from the advantages or potential of the cells obtained, methods, and conditioned media resulting from the invention.

### DETAILED DESCRIPTION OF THE INVENTION

When developing the present invention, the cardiomyogenic and angiogenic potential of three structures that form part of the placenta was studied. It was studied angiogenic potential cardiomiogénicos, the differences between the placental areas and the area having the placental MSCs with more cardiomyogenic and angiogenic potential compared to bone marrow MSCs. To carry out this development induction into cardiac lineage with 5-azacytidine was performed and evaluated by RT-qPCR expression of NKX2-5, GATA-4, MYH7b, GJA1 and TNNT2 genes involved in cardiomiogénesis. The presence of connexin 43 and cardiac troponin was determined by indirect immunofluorescence. To assess the potential of angiogenesis it was developed an in vitro angiogenesis assay using Matrigel.

It was found a greater cardiomyogenic and angiogenic potential in mesenchymal stem cells from the chorionic cells, CHOR-MSCs, compared to decidual, DEC-MSCs, and umbilical cord, UC-MSCs, being the cardiomyogenic potential of CHOR MSCs higher to the levels obtained in bone marrow MSCs. About the potential of MSCs to form chorion tube-like structures, CHOR-MSCs demonstrate a greater potential that the bone marrow derived MSCs, besides presenting a higher potential with respect MSCs from umbilical cord and decidua.

The paracrine effects of MSCs involved in the induction of angiogenesis was also demonstrated, since the supernatant of MSCs (or conditioned medium) induced tube-like structures formation in vitro in an endothelial cell line. The above evaluation established that the supernatant of CHOR MSCs induces an increased tube-like structures formation in vitro.

The results obtained in the development of the present invention indicate that the best placental tissue to be used as a source of MSCs for cell therapy (e.g. heart failure) is the Chorion-derived MSCs cells.

According to the above observations, MSCs isolated from chorionic tissue according to the present invention have a high performance over the other sources of mesenchymal stem cells derived from the placenta, exhibiting higher potential for in vitro cardiomyogenic and angiogenic than bone marrow-derived MSCs.

Moreover, by the standards of International Society for Cell Therapy, ISTC, is not possible to perform a therapy mixed from two individuals, which in the case of placental origin is of both fetal and maternal cells. Consequently, a major advantage of the MSCs isolated from chorionic tissue according to the present invention lies in the favorable regulatory implications due to the absence of maternal contamination with the fetal cells.

Clinical trials are focusing on establishing the MSCs therapeutic benefit for degenerative diseases such as cardiac insufficiency. The traditional source is bone marrow; however, there are many limitations for its use, such as the low initial MSC quantity, invasiveness when getting a sample, and complications with the donor age. Delivered human placenta is considered a source of abundant maternal and fetal MSCs; this thesis sets forth that MSCs isolated from chorion, umbilical cord, and decidua have different in vitro cardio-myogenic and angiogenic potential.

The aim of this invention was to establish differences with regard to MSC cardio-myogenic and angiogenic potential, obtained from 3 areas of delivered human placenta: chorion, decidua, and umbilical cord. At the same time, it was established whether the placenta area with MSCs with the higher cardio-myogenic and angiogenic potential also shows a potential equal or higher than MSCs from bone marrow in order to choose suitable tissue and obtain the more effective placental tissue to be used in cardiac regeneration therapies and this establishes that the placenta represents a more accessible and efficient source than bone marrow.

Mesenchymal stem cells from the chorion plate (CHOR-MSCs) have a higher in vitro angiogenic potential compared to umbilical cord, decidua, and bone marrow MSCs.

Unlike the previously mentioned studies, the experimental model in this work did not include pre-treatment with inductor factors to endothelial lines, which is an important detail and would indicate that preparing MSCs for a revascularization therapy implies procedures with little intervention, unlike genetic manipulation, using factors that are normally part of the organism and are involved in the angiogenesis positive regulation, such as the vascular endothelial growth factor (VEGF), fibroblast growth factor (FGF), hepatocyte growth factor (HGF), and the angiopoietin (Ang). Some studies have demonstrated that pre-conditioning the MSCs to the said factors before being included in cardiac therapies increases benefits for ischemic hearts.

It is necessary to highlight that human placenta vascularization is the result of capillary blood vessels forming by pluripotent mesenchymal cells that form the original chorion, and correspond to MSCs present in the delivered placenta.

In delivered human placenta, decidua has been largely replaced by chorion and, therefore, with regard to abundance, it is possible that the delivered placenta is not a source of excellence to get DEC-MSCs. At birth, a large part of the decidua remains adhered to the mother endometrium, which later comes out in the form of an after delivery hemorrhagic fluid, which is possibly an abundant source of DEC-MSCs. The above sets forth that, regarding abundance, delivered human placenta (number of cells obtained from the initial sample) is the best source of CHOR-MSCs and UC-MSCs. This can be demonstrated by calculating the percentage of fetal cells in samples isolated from decidua by genotyping (gen XX or XY) in placentas obtained when the fetus is male.

CHOR-MSCs supernatant, under normoxia and hypoxia conditions, had a higher potentiating effect on the HUVEC angiogenesis capacity compared to the sUC-MSCs and sDEC-MSCs effect. According to the total loops parameter, the CHOR-MSCs supernatant, when submitted to hypoxia for 48 h, had a higher positive effect than the EGM media on the HUVEC angiogenic potential. Scientific studies have established that MSCs release a series of soluble factors such as VEGF, FGF, HGF, and stem cell factor, SCF, which have been involved in neo-vascularization induction. VEGF, especially, has been proposed as a key factor in the MSCs cardiac. CHOR-MSCs have higher HGF expression levels compared to other placenta and bone marrow sources. Due to the above, it has been set forth that calculation and quantification of soluble factors released by placenta and bone marrow, using ELISA tests, could demonstrate factors released by CHOR-MSCs through a paracrine medium.

The present invention allowed establishing that hypoxia increases secretion of factors involved in angiogenic induction. These observation can be supported by other scientific studies that have demonstrated that MSCs submitted to hypoxia express higher levels of factors favorable to survival and angiogenesis, including the factor inducible by hypoxia 1 (HIF-1), angiopoietin 1, and VEGF in vitro, and the transplant of these MSCs pre-conditioned to hypoxia improves cardiac function, very probably due to an increase in post myocardial infarct (MI). In vivo angiogenesis trials have also demonstrated that the use of a conditioned medium of MSCs submitted to hypoxia increases revascularization in the subcutaneous matrigel implant. The previous results match researches carried out with cardiac isquemia in animal models, where it has been established that MSCs participate in angiogenesis, improving cardiac function.

### Conditioned Media

Results obtained in the present invention indicate that CHOR-MSCs have a higher differentiation potential towards cardiomyocite and a higher angiogenic potential.

Another approach that has had interesting results is the use of factors to pre-treat MSCs before introducing them. These factors would allow intensifying their therapeutic properties, increasing migration capacity towards the corresponding tissue, as well as cell survival.

CHOR-MSCs conditioned media, whether under hypoxia or normoxia conditions, have an important commercial value as they can be used as a complement for the culture of different cells in vitro, as well as an in vivo effect on cell growth and regeneration with application not only on degenerative diseases, but also as a product for sportspeople muscle regeneration and development.

The advantages of the conditioned media over the use of synthetic growth factors such as VEGF, FGF, HGF and angiopoietins, etc., in addition to a lower cost, is that the injection of a composition based on this conditioned medium remains effective in vivo over a longer period of time, without requiring re-injected as would occur with the use of a composition of synthetic factors.

Conditioned media can be used in academic research for the culture of different types of stem cells, for instance, for induced pluripotent cells (IPS), embryo stem cells (ES), and hematopoietic stem cell (HSC) cultures (ANLI OUYANG, stem cells 2007).

On the other hand, skeleton muscle injuries are very common and represent up to 35-55% of all sports injuries, and are possibly related to muscle-skeleton traumas. These injuries result in the forming of fibrosis that could lead to the development of painful contractures, they increase the risk of repeated injuries and limit the capacity to return to a functional level. Indirect use of MSCs is proposed as a conditioned CHOR-MSCS medium, containing different growth factors and with a regenerative capacity that can improve local supply in the injury area by promoting angiogenesis as demonstrated in this invention.

Moreover, cosmetic companies propose products for cosmetic use (acne, skin rejuvenation, anti-wrinkle). The conditioned media contains proteins that have been secreted by the adipose tissue isolated cells.

### Description of Cell Types Used

For the present invention, 4 MSCs sources were analyzed: on the one hand, the 3 areas forming the placenta: chorion, decidua, and gelatin of Wharton and, on the other hand, it was necessary to obtain MSCs from bone marrow, as this is the most studied source for in vitro as well as for in vivo assays, and it represents the reference source (“Gold Standard”).

Also, an endothelial cell line was used as a positive control for the forming of in vitro tube-like structures, which was also used to assess the effect of factors released by the MSCs on their angiogenic potential. The following is a brief description of the characteristics of cell types used in this study.

### Mesenchyme Stem Cells (MSCs)

Human placenta and bone marrow, from which MSCs for the study were isolated, were obtained with the corresponding informed consent, approved by Clínica Dávila Ethics Committee. Donated placenta and bone marrow were processed at miRNA Research and Development Laboratory and stem cells were processes at Universidad de los Andes Cells for Cells.

MSC primary cultures were obtained from placenta chorion plate (CHOR-MSC), decidua (DEC-MSC), and umbilical cord gelatin of Wharton (UC-MSC). Placenta MSCs, as well as those obtained from bone marrow (BM-MSCs), were characterized at the same laboratory, for which their mesenchyme phenotype was determined by assessing a classic surface marker panel for MSCs. The in vitro differentiation potential was also assessed, and their capacity to adhere to plastic was confirmed. One important parameter when seeking an alternative source for cell therapy is the cells obtained present an optimal proliferation rate under standard culture conditions, for which reason this characteristic was established for all sources under study. All these results may be observed by referring to the Examples. Results indicate that MSCs used in the present invention comply with the study basic considerations.

Human Endothelial Cells from the Umbilical Cord Vein (HUVEC)

In order to carry out in vitro angiogenesis assays, the HUVEC (ATCC® CRL1730™) cell line was used, with the corresponding characterization and quality certification. These are cells isolated under standardized conditions. From the human umbilical cord vein vascular endothelium, they adhere to plastic and proliferate fast in the correct culture medium.

Obtaining MSCs from Placenta Tissues and Bone Marrow

The work plan developed in the Research and Development Laboratory of miRNA and stem cells from Universidad de los Andes Cells for Cells laboratory to obtain placenta and bone marrow donors allowed the acquisition of enough quantity of biological triplicates (i.e. 3 different donors) and MSCs isolated under standardized culture conditions ruled by good manufacturing practices (GMP) standards. After-birth tissue handling and bone marrow blood, in addition to cryopreservation of primary cultures obtained from different sources were the responsibility of the cell therapy unit at the same laboratory.

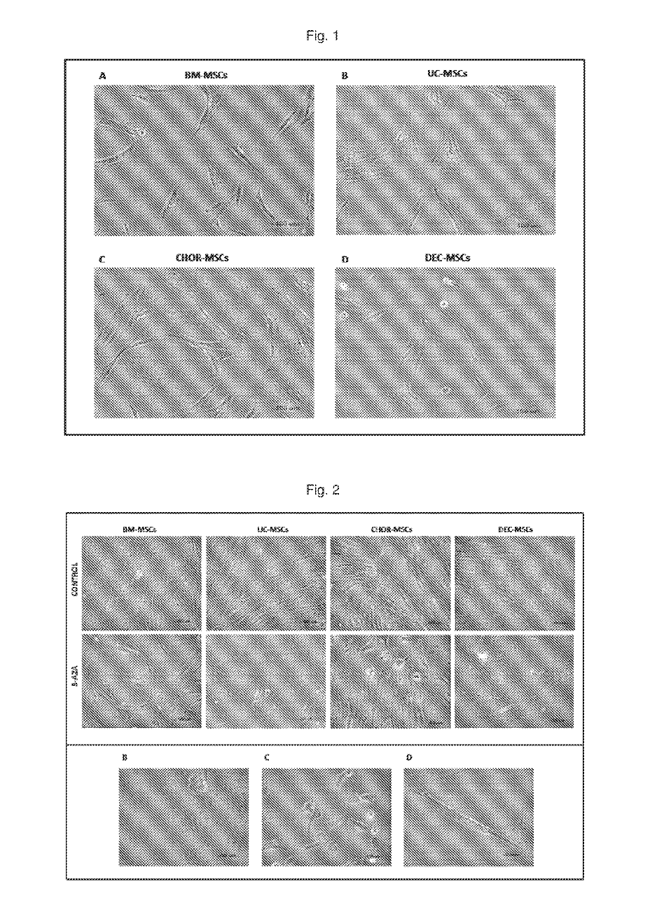

Cells were obtained from cryo-preserved primary tissue. Once MSCs were unfrozen for expansion, they grew adhered to plastic (FIG. 1), and it was possible to obtain a large number of cells between passages 2 to 6 in order to carry out cardio-myogenic induction experiments and angiogenesis trials. In culture, the placenta and bone marrow MSC populations presented heterogeneous fibroblastoide morphology.

MSCs Cardio-Myogenic Induction with 5-Azacytidine

In order to calculate cardiac differentiation capacity of chorion, umbilical cord, decidua, and bone marrow MSCs, induction with 5-aza was carried out. This experiment allowed identifying, from the 3 placenta sources, the source with the highest in vitro cardio-myogenic potential and, at the same time, evaluating whether this potential was equal or higher than that shown by bone marrow MSCs.

### MSCs Morphology During Cardiac Induction

In order to establish morphology changes occurred in MSCs treated with 5-aza, photographs were taken daily for later analysis. 3 to 5 days after induction with 5-aza, differences were noted in controls regarding proliferation of treated MSCs, which is evidenced by observing photographs confluence in FIG. 2. After 20 days, confluence in both conditions was quite similar and reached 100%. In some dishes, it was common to find a reduced percentage (10%) of cells with vacuoles inside them. On the other hand, it was also noted that a large percentage (approximately 70%) of cells adopted a rounded and flat morphology, while other presented a longish tubular morphology which, in some cases, were multi-core. These observations in culture were complemented with an assessment at molecular level analyzing the expression of genes associated to the heart line in order to demonstrate the cardiac differentiation.

### Quantification of Relative Expression of Genes Involved in Cardio-Myogenesis Through RT-qPCR.

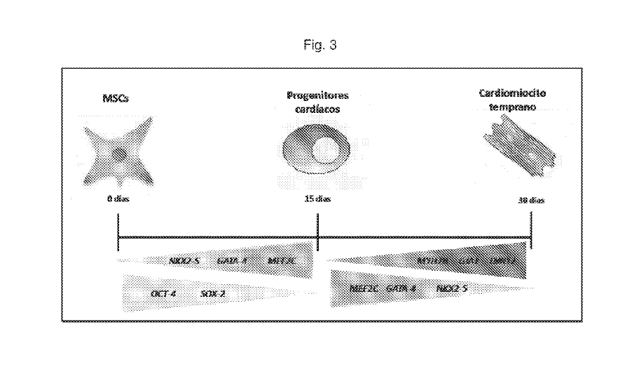

In order to establish the differentiation potential to cardyomyocite, the relative expression of genes involved in cardio-myogenesis was assessed after induction with 5-aza using the RT-qPCR technique and later data analysis using the CT (cycle threshold) double delta analysis. Calculation of expression profiles allowed comparisons between the MSC sources under study, establishing a direct relation between a gene highest relative expression and its highest differentiation potential. FIG. 3 shows a scheme based on publications that identify genes analyzed in this research and determine an expression profile along a period of time.

In order to determine whether starters designed to assess the relative expression of genes involved in cardio-myogenesis were optimally operating in the RT-qPCR reaction, the starters efficiency assessment was carried out with the corresponding cDNA (for human heart cDNA cardiac markers and for the MSC cDNA standardizing gene).

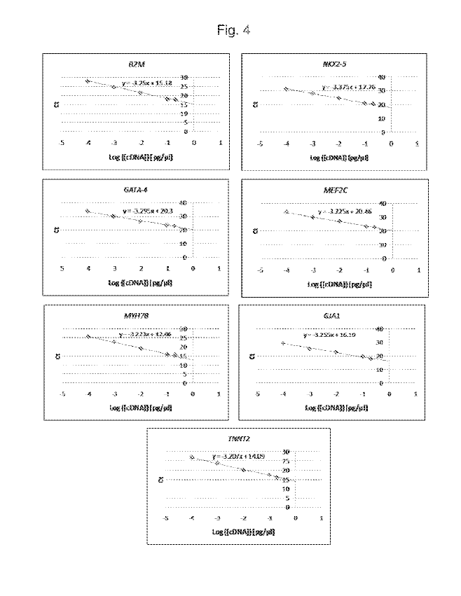

The RT-qPCR technique and the result analysis, using the CT double delta method, allowed identifying a significant increase in the relative expression of all genes associated to cardio-myogenesis in all MSC sources treated with 5-aza, compared to control after 30 days of culture after induction (see FIGS. 4 and 5).

Relative expression levels of NKx2.5 and GATA-4 transcription factors have a scale approximately twice lower than genetic markers involved in a more advanced differentiation status, such as MYH7B, GJA1 and TNNT2 genes. Comparative analyses of relative gene expression of MSC sources place CHOR-MSCs as the source with the highest levels in all genetic markers regarding MSCs from umbilical cord and decidua, with approximately twice NKX2-5, GATA-4, MEF2C, MYH7B, GJA1, and TNNT2. However, in connection with BM-MSCs, it is not significantly different in its expression levels, except for genes MEF2C and GJA1, where the CHOR-MSCs relative expression is twice the bone marrow MSCs.

TABLE 2
Nucleotide Sequences of Designed Starters,
RT-qPCR Product Melting Temperature (Tm)
and Efficiency Percentage in the Reaction.

| | | Ampli- | Effici- |
|---|---|---|---|
| | | con | ency |
| | Starters | Tm | (%) |
| B2M | F: 5′TCAGGTTTACTCACGTCATCC3′ | 80.6 | 103.09% |
| | R: 5′ACACGGCAGGCATACTCATC3′ | | |
| GATA- | F: 5′AAACGGAAGCCCAAGAACCT3′ | 87.1 | 101.13% |
| 4 | R: 5′ACTGAGAACGTCTGGGACAC3′ | | |
| NKX2- | F: 5′TGTCCACGCTGCATGGTATC3′ | 86.6 | 97.83% |
| 5 | R: 5′GATCACTCATTGCACGCTGC3′ | | |
| MEF2C | F: 5′CCAACTTCGAGATGCCAGTCT3′ | 83.2 | 104.2% |
| | R: 5′GTCGATGTGTTACACCAGGAG3′ | | |
| MYH7B | F: 5′GCAATAAAAGGGGTAGCAGAGC3′ | 81.6 | 104.3% |
| | R: 5′GACTCCCCAAGTTCACTCACAT3′ | | |
| TNNT2 | F: 5′CTGGCCATTGACCACCTGAA3′ | 82.1 | 105.03% |
| | R: 5′GCTGCTTGAACTTCTCCTGC3′ | | |
| GJA1 | F: 5′TCTCTCATGTGCGCTTCTGG3′ | 80.6 | 103% |
| | R: 5′TGACACCATCAGTTTGGGCA3′ | | |

Detection of Proteins Involved in Cardiac Differentiation in MSCs Treated with 5-Azacitidine.

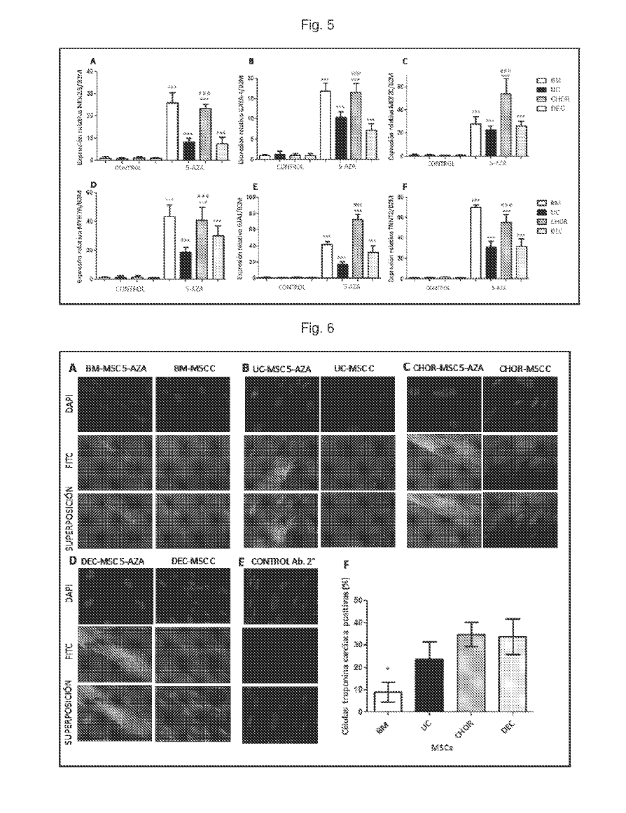

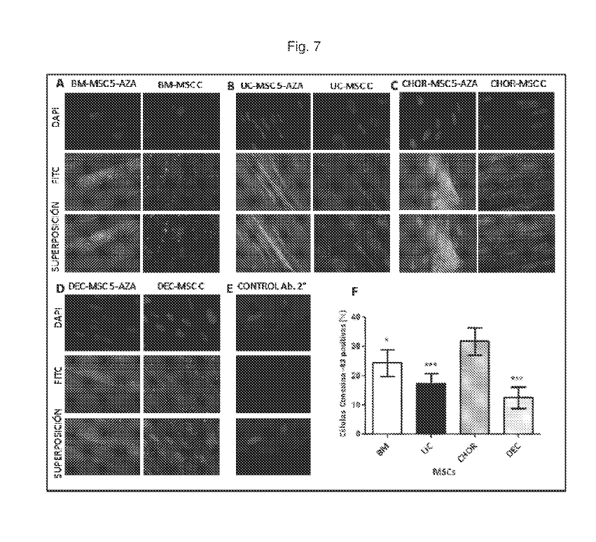

In order to determine whether the molecular marker profile expressed in responses to treatment with 5-aza was a protein expression, IFI assays were carried out, where cardiac troponin (FIG. 6) and conexine 43 (FIG. 7) responses were assessed.

Both proteins were viewed through fluoresce in isothiocynate fluorochrome (FITC) associated to the secondary antibody, which recognized primary antibodies specific for cardiac troponin and conexine 43 (CX-43). MSCs considered positive differentiate from the technique non-specificity through the fluorescence intensity. No computer analyses were carried out to determine cell distribution and protein ultra-structure.

The percentage of positive cells for cardiac troponin was significantly higher for decidua and chorion MSC, reaching approximately 35%, which represents 3 times more than BM-MSCs and twice more than UC-MSCs. For conexine 43 marking, a higher number of positive cells was established in CHOR-MSCs compared to UC-MSCs, DEC-MSCs, and BM-MSCs, reaching 10% more than UC-MSCs and 20% more than DEC-MSCs.

Detection of proteins related to heart line allows establishing the 5-aza cardiac induction effect and determining that transcription and gene factors assessed through RT-qPCR translate their expression to cardiac proteins that perform important functions in mature cardiomyocytes. One of the proposals to explain the MSC repair mechanism in hearts with an infarct is its tissue graft and later differentiation to cardiomyocyte

### Assessment of MSC In Vitro Angiogenic Potential

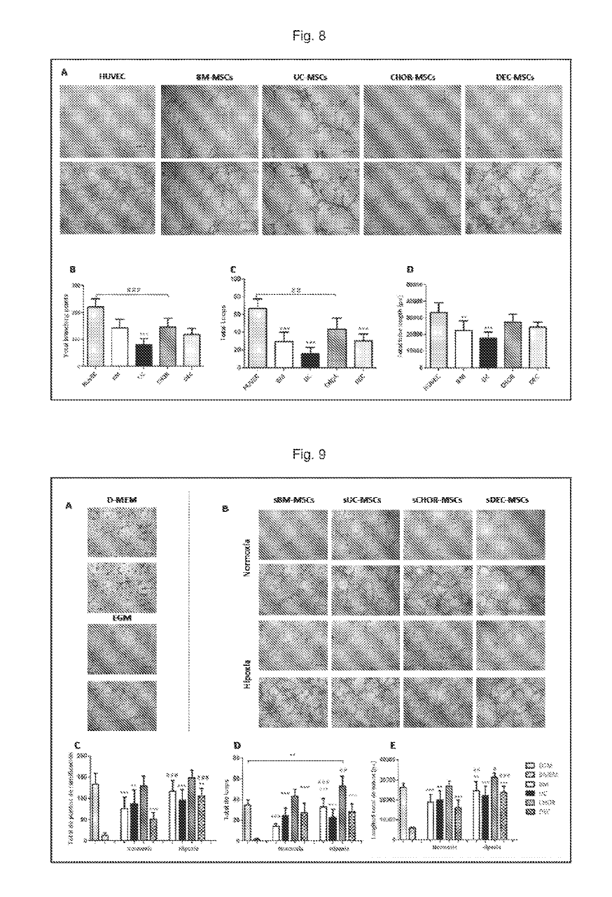

In the present invention was established whether placenta and bone marrow MSCs had the capacity to form in vitro tube-like structures when cultured on matrigel, which is a commonly used method to assess angiogenic potential in vitro. With this assay, it was possible to observe the forming of tubes by all placenta and bone marrow MSCs; however, interesting differences between the sources analyzed occurred (FIG. 8).

In the three parameters used to quantify angiogenesis potential in vitro (total tube length, total loops formed, and total ramification points), endothelial cells showed a tendency to a higher potential, reaching at once significant differences compared to all MSCs under study regarding the forming of loops and total ramification points. Comparison of angiogenic potential between different MSC sources shows that, according to loop forming, CHOR-MSC have a potential twice higher than DEC-MSCs, UC-MSCs, and BM-MSCs, which is not very evident according to the total ramification points, where CHOR-MSCs show a significantly higher potential (1.5 higher) only with regard to UC-MSCs. There is a tendency to a higher angiogenic potential of DEC-MSCs compared to UC-MSCs. This places the studied MSCs in an angiogenic potential range of the following order CHOR-MSCs>BM-MSCs>DEC-MSCs>UC-MSCs.

One of the theories proposed to justify recovery when therapies with MSCs are administered, is the MSCs capacity to get into vessels and start forming new tube-like structures. However, there this is also evidence of potential benefit from factors releasing the MSCs and promoting, through a paracrine means, the ischemic tissue re-vasculogenesis.

### Assessment of the MCS Supernatant Effect on the HUVEC In Vitro Angiogenic Potential.

In order to establish whether placenta and bone marrow MSCs release factors that are media for angiogenesis activating processes, an in vitro angiogenesis assay was carried out using matrigel reduced to factors in order to assess supernatants obtained from MSCs under hypoxia conditions to simulate ischemic and normoxia conditions as control.

All placenta and bone marrow MSC supernatants, under hypoxia as well as under normoxia conditions, induce the forming of in vitro tube-like structures (FIG. 9). Also, under both conditions, it was detected that CHOR-MSC supernatant capacity to induce angiogenesis is twice the average, compared to UC-MSCs, DEC-MSCs, and BM-MSCs supernatants. It is important to note that, according to the parameter that measures total loops formed, the supernatant of CHOR-MSCs submitted to hypoxia for 48 h has a potential to induce angiogenesis that is 1.5 times higher, compared to the positive control media and endothelial growth media (EGM). The potential to induce angiogenesis of the supernatant of all MSCs submitted to hypoxia, except for UC-MSCs, was significantly higher compared to the normoxia condition in the case of supernatant of MSCs from bone marrow. According to the 3 parameters considered, an increase of twice the value obtained under hypoxia conditions was observed, compared to normoxia. For MSCs derived from chorion and decidua, an increase in the hypoxia condition of 1.5 and 2 times, respectively, was observed in at least two of the three parameters, compared to supernatants obtained under normal oxygen conditions (FIG. 9 C, D, E).

### EXAMPLES

### Example 1

### Cell Culture Conditions

Primary MSC and HUVEC cell line cultures we worked with, were kept in DMEM (Dulbecco's Modification of Eagle's Medium, Gibco USA), complemented with 10% of bovine fetal serum (FBS, Lonza USA) previously deactivated in a thermo-regulating bath during 30 minutes at 56° C., with added L-glutamine 200 Mm (Gibco, USA) at 1% in the final medium volume. Furthermore, the culture medium was added a mix of penicillin and streptomycin (Gibco, USA) at 100 U/ml and 100 ug/ml, respectively, with 1% concentration in the final volume. Every 2 to 3 days the medium was aspired, cells were washed with PBS 1× adjusted to pH 7.4 (Life Technologies, USA), and fresh culture medium was added. Also, it was always ensured that cell confluence in the container did not exceed 70%. Cells were kept in an incubator (Thermo Scientific Series 8000 WJ, USA) humidified at 37° C. in a 5% CO₂ environment. All cell culture procedures were carried out under a laminar flow bell, biosafety II (ESCO, USA). To observe cell cultures and cell counting, the optical microscope Olympus CKX41 was used, and photographs were taken with an Olympus U-TV1X-2 camera.

### Example 2

### Tripsinizing and Cell Count

When the cell count reached 60-70% confluence, cells were detached through incubation at 37° C. with Trypsin-EDTA (Gibco, USA) during 5-10 minutes; then trpsyn was deactivated and 10% FBS DMEM was added. This mixture was collected in a Falcon-type tube and centrifuged at 300×g during 6 minutes; the supernatant was discarded and cells were re-suspended in 1 ml 10% FBS DMEM. An aliquot was taken from the cell suspension and mixed with trypan blue in a proportion of 1:1, and 10 μl were loaded in a Neubauer chamber or hemocytometer. Trypan blue penetrated the cells only when the cell membrane was deteriorated. In order to calculate the number of feasible cells, cells not tinted with trypan blue were counted in each one of the Neubauer chamber quadrants; the average of this number was found, and the number of live cells per ml was calculated according to the following formula:

Number of feasible cells/ml: PC×FD×10.000

Where, PC: Average live cells and FD: Dilution factor (2)

This calculation allowed establishing the volume to be removed from the initial cell re-suspension in order to plant the wished amount in the new container. In the case of expansion, cell cultures were planted at an initial density of 3000 cells per cm². Plastic containers were used during cell expansion and experiments were of the BD Biosciences brand; different dimensions were used, depending on the culture purpose.

### Example 3

### Cell Freezing and Thawing

For cryopreservation, cells were tripsinized and counted as indicated in section 3. Cells were taken to a concentration of 1 million per ml in the freezing medium, formed by 90% deactivated FBS (Gibco USA) and 10% cryoprotector agent DMSO (Di-metil-sulfoxide, Sigma). Cell suspension was distributed at a ratio of 1 ml per cryotube, and those viable were frozen in an isopropyl alcohol container which, at −80° C., allowed temperature to decrease 1° C. per minute, a progressive cooling necessary to the cells successful freezing. After 4 hours, cells were stored in a liquid nitrogen tank (Thermo Scientific 8146, USA).

For thawing, cells were transferred from the liquid nitrogen tank to a thermo-regulated bath at 37° C., for quick thawing. Cell suspension was transferred to a 15 ml tube of the Falcon type containing the culture medium, and centrifugation at 300×g was carried out during 6 minutes. The supernatant was discarded, which contained the freezing medium, cells were re-suspended in 1 ml of culture medium at 37° C., and then counted as detailed in section 3. Then, cells were planted at a density of 3000 cells per cm2. After 4 hours, the majority of cells were already adhered to the plastic surface. The following day, the medium was replaced in order to eliminate dead cells.

### Example 4

### Mycoplasma Control in Cell Cultures

Mycoplasma contamination is not visually perceived in cell cultures, but it could affect experimental results. The mycoplasma routine test was carried out in all types of cell in culture using EZ-PCR Mycoplasma Test Kit (Biological Industries, USA). This test is based on the detection of microorganisms by PCR using specific primers. For the PCR, the cells culture medium was used after at least three days in a condition of over-confluence and absence of antibiotics.

When mycoplasma was detected through a positive PCR, cells were decontaminated with a treatment with Byomic 1 (Biological USA) at 1% in the culture medium during 4 days, and then in Byomic 2 (Biological USA) at 1% in the culture media during 3 days, this alternation was performed during 3 weeks. Later, the presence of mycoplasma was assessed again in order to verify the treatment effectiveness.

### Example 5

### Cardio-Myogenic Induction Test with 5-Azacitidin

In order to assess chorion, decidua, gelatin of Wharton, and bone marrow MSCs cardio-myogenic potential, 3000 cells per cm² of each one of the MSCs were planted in biologic triplicate (3 different donors) in a plate of 6, leaving 3 bowls for induced condition and other 3 for control condition. This method was developed to obtain cells for assessment through RT-qPCR. However, in order to assess protein expression through indirect immunofluorescence (IFI), cells were planted on a 24 cm circular glass cover placed on a 24-bowl plate, leaving 12 bowls for induced condition and 12 bowls for control condition.

For induction, MSCs we incubated with DMEM, 2% FBS, 1% Pen/Strep, 1% L-glutamine, supplemented with 10 μM 5-zacitidine (Sigma, USA) during 24 hours, and kept in a humidified and thermo-regulated incubator at 37° C. and 5% CO₂. Later, they were removed from the induction medium, cells were washed three times with PBS 1× adjusted to ph 7.4 (Life Technologies, USA), and cultured in a maintenance medium during 30 days, being renewed every three days. The control condition corresponded to cells submitted to DMEM 2% FBS with no 5-aza during 24 hours, and then cultured in maintenance medium during 30 days.

### Example 6

### Assessment of Genic Expression Through RT-qPCR

The polymerase chain reaction in real time, preceded by reverse transcription (RT-qPCR), was based on quantification kinetics that allowed calculating the messenger RNA through the recording of PCR product forming in real time. This was the quantification, during the reaction exponential stage, of amplified PCR products and labeled with fluorochrome. In this work, a relative quantification of genes of interest was carried out, i.e. a gene of interest expression level is obtained compared to another one that keeps its expression constant and is called standardizing gene; therefore, in this thesis human gene B2M was selected as standardizing.

Obtaining Complementary DNA (cDNA)

From each cell sample, the total RNA was extracted using “RNeasy Plus Mini Kit” (Qiagen, USA), according to the manufacturer instructions. RNA was quantified and its purity was calculated with a spectrophotometer using the NanoDrop (Thermo Scientific, USA) equipment according to the manufacturer instructions. In order to carry out the cDNA synthesis, RNA 1-2 μg were used. Prior to the cDNA synthesis, each RNA sample was treated with DNAasa I (RNase-Free) (Appplied Byosistem, USA); 1 μl Buffer DNAasa I 10× and 1 μl DNAasa I (2 U) were briefly used with DEPC water (Invitrogen, USA); a final volume of 10 μl was adjusted and incubated at 37° C. during 30 minutes. After incubation, 1 μl EDTA 50 mM (Invitrogen, USA) was added and incubated again at 75° C. during 10 minutes. Later, each tube was placed on ice and added 2 μl of Primer Random Nonamers (0.5 μg/μl) (Promega, USA), 4 μl dNTP mix (10 Mm) (Promega, USA), and the volume necessary of DEPC water in order to get a mixture in a final volume of 17 μl; then, this was incubated for 5 minutes at 72° C. Once incubation was completed, tubes were placed on ice again, 2 μl Buffer RT 10×, 1 μl de RNAse inhibitor, and 1 μl de M-MuLV Reverse Transcriptase (200 μ/μl) (Promega, USA) were added in a final volume of 21 μl; the mixture was incubated at 42° C. for 1 hour. Once the incubation was completed, synthesized cDNA was immediately used for RT-qPCR reaction or kept at −20° C. For different incubations, thermal cycler Swift™ MaxPro (Esco, Singapore) was used.

### Design and Efficiency of Starters for RT-qPCR

In order to calculate cardio-myogenic potential, the expression of genes associated to early cardiac differentiation was used, such as transcription factors GATA-4, NKX2.5 and MEF2C. Also, late differentiation genes were used, such as MYH7B, TNNT2, and GJA1. Functional descriptions for each gene used are shown in Table 3. The starters design was carried out using the program Primer-Blast and the messenger RNA described in the National Center for Biotechnology Information (NCBI) database. Once the starter sequences for each gene were calculated, they were synthesized by the company Integrated DNA Technologies (IDT).

Lyophilized starters were reconstituted with DEPC water, leaving them at a concentration of 10 μM. Efficiency percentage was calculated for each starter by carrying out serial dilutions based on 10 (1:10, 1:100, 1:1000, 1:10000) of cDNA. From each dilution, 2 μl were charged to the RT-qPCR reaction. For starters related to heart line, synthesized cDNA was used based on RNA “Human Heart Total RNA 100 ug” (Ambion AM7966, USA) and, for the standardizing gene, BM-MSCs cDNA was used. The efficiency percentage was calculated according to the formula: ((10̂(−1:mx))−1)*100), where mx is the value of the slope originated from the Log 10 graph on cDNA concentration v/s the “cycle threshold” value (CT).

TABLE 3
Description of genes used to assess cardio-myogenic differentiation through RT-qPCR

| Gene | GenBank | | |
|---|---|---|---|
| symbol | access number | Gene name | Description |
| B2M | NM_004048.2 | Beta-2-microglobulin | It codifies for the beta chain of the major |
| | | | histo-compatibility complex class I. |
| GATA-4 | NM_002052.3 | Homo sapiens GATA | It codifies for the transcription factor that |
| | | binding protein 4 | recognizes DNA GATA motif and |
| | | | regulates the expression of genes |
| | | | involved in embryogenesis and in |
| | | | myocardium function and differentiation. |
| NKX2-5 | NM_004387.3 | Homo sapiens NK2 | This gene contains a homeobox 5 |
| | | homeobox 5 | domain and codifies the expression of |
| | | | transcription factor NKx-2.5 involved in |
| | | | the forming, development, and |
| | | | functioning of the heart. |
| MEF2C | NM_002397.4 | Homo | It codifies for the MEF2C transcription |
| | | sapiens myocyte | factor, necessary for the correct |
| | | enhancer factor 2C | development of the cardio-vascular |
| | | | system. |
| MYH7B | NM_020884.3 | Homo sapiens myosin, | This gene codifies a heavy chain of |
| | | heavy chain 7B, | myosin II. The chain includes a globular |
| | | cardiac muscle, beta | domain which catalyzes ATP hydrolysis |
| | | | and interacts with actin. |
| TNNT2 | NM_000364.3 | Homo sapiens | This gene codifies for the troponin |
| | | troponin T type 2 | tropomyosin union complex sub-unit, |
| | | (cardiac) | located in the thin filament of striated |
| | | | muscles and regulates muscle |
| | | | contraction in response to alteration in |
| | | | intra-cell calcium ions concentration. |
| GJA1 | NM_000165.3 | Homo sapiens gap | This gene belongs to the conexine gene |
| | | junction protein, alpha | family. Codified protein is the main gap |
| | | 1, 43 kDa | union protein in the heart, which is |
| | | | considered to have an essential role in |
| | | | the heart synchronized contraction. |

### RT-qPCR, Reaction, Analysis and Quantification

RT-qPCR reaction used to analyze gene expression in MSCs under study was carried out under the following conditions: 3.31 μl DEPC water, 0.375 μl Forward primer, 0.375 Reverse primer, 6.25 μl Brilliant II SYBR® Green QPCR Master Mix (Invitrogen, USA), 0.187 μl Rerence Dye (Invitrogen, USA), and 2 μl de cDNA obtained from 1-2 μg dRNA in 12.5 μl final volume.

The RT-qPCR reaction thermal profile was carried out in a thermo cycler Mx3000P Real Time PCR System (Stratagene, USA); temperature cycles were as follows: 1 initial cycle at 95° C. for 10 minutes, followed by 40 cycles of 30 seconds at 95° C., 30 seconds at 60° C., 45 seconds at 72° C., and one final cycle of 1 minute at 95° C., 30 seconds at 55° C., and 30 seconds at 95° C. In order to obtain relative changes quantification in the gene expression, the 2^{-ΔΔCT} method was used, where “y” is the relative expression, “CT” is the cycle in the amplification reaction where fluorescence increases in an exponential way with regard to basal fluorescence, and “−ΔC(t)” is the results of subtracting the CT value from the constitutive gene for each sample.

### Example 7

### Detection of Protein Expression Related to Heart Line Using Indirect Immunofluorescence (IFI)

MSCs were planted on a 12 mm circular cover glass (EDLAB, Germany) to carry out a trial with 5-azacitidin (5-aza). After 30 days induction, the 24-bowl plate containing the cover glasses was removed from the incubator, the culture medium was aspirated and washed with PBS 1×pH 7.4 (Gibco, USA). MSCs were fixed with paraformaldehyde (PFA) at 4% for 15 minutes at room temperature; then, they were washed twice with PBS 1×. Later, cells were made permeable with 0.25% tritonX-100 in PBS 1× for 10 minutes at room temperature, and then washed during 5 minutes with PBS 1×. Following, they were blocked with a solution of 1% BSA in PBS1× with 0.25% tritonX-100 during one hour at room temperature, leaving the cover glasses inside a humid container.

After the incubation time in blocking solution, cells were incubated for one hour at 4° C. with primary antibodies corresponding to Anti-Cardiac Troponin T antibody (ab45932 Abcam, USA), and Anti-Connexin 43/GJA1 antibody (ab11370 Abcam, USA), both diluted in 1% BSA in PBS-0.25% Triton100, at a proportion of 1:200. After the incubation time with primary antibodies, cells fixed on the glass covers were washed three times with PBS 1× and incubated for one hour at 4° C. with secondary antibody Donkey anti-Rabbit IgG H&L (FITC) secondary antibody (ab6798 Abcam, USA) diluted in 1% BSA in PBS-0.25% Triton100 at 1:2000 concentration. Preparations were protected from light, always including a control and using secondary antibody only. Later, cells were washed three times with PBS 1× and incubated with DAPI (Invitrogen, USA) at 1 ug/ml concentration during 1 minute, and washed once with PBS 1×. Glass covers were mounted (with the cells surface downwards) on a holder using the mounting solution Fluorescence Mounting Medium (Dako, USA).

The previous preparations were observed under a NIKON ECLIPSE TE2000-U fluorescence microscope, and photographed with a Nikon Sight DSU2 digital camera. Three independent experiments were carried out in triplicate and, for later positive cell number quantification, three photographs were taken for each condition. In order to calculate the number of positive cells, the said images were analyzed, counting the number of total nucleus per image with regard to the number of positive tinted cells.

### Example 8

### Assessment of MSCs Angiogenic Potential Using the In Vitro Angiogenesis Assay

In order to calculate the placenta and bone marrow MSCs in vitro angiogenic potential, a preparation extracted from a mouse sarcoma and called Engelbreth-Holm-Swarm (EHS) was used. This is a protein-rich tumor from the extracellular matrix. The preparation used was BD Matrigel matrix (354234 BD, USA), applied according to the manufacturer instructions, with some modifications. Briefly, before using matrigel, pipette tips and the 24-bowl plate were thawed at 4° C. during the entire night as, at the coating moment, matrigel could not exceed 20° C., as it gels very quickly at the said temperature. 300 μl matrigel per bowl were dosed, and the plate was incubated for 40 minutes at 37° C.

After the incubation, the corresponding cells were planted on matrigel at a density of 60,000 cells per bowl. For this assay CHOR-MSCs, DEC-MSCs, UC-MSCs, and BM-MSCs were used, and HUVEC as positive control. Cells were incubated under normal culture conditions (37° C., 5% CO2) and in an endothelial growth culture medium (EGM), making sure that its distribution on the entire matrigel surface was homogeneous. The assay was checked after 5 hours culture, taking 4× photographs through optical microscope Olympus, model CKX41 and camera Olympus U-TV1X-2.

### Angiogenic Potential Quantification

In order to carry out the quantitative analysis of angiogenic potential, three independent experiments were carried out in triplicate for each MSCs source and for HUVEC cell line; three photographs were taken of each condition. Photographs were analyzed with the program WimTube (Wimasis GmbH, Munich, Germany), which allowed comparing sources under study regarding tubes total length, total ramification points, and total loops formed.

### Example 9

### Assessment of the MSCs Supernatant Effect on HUVEC In Vitro Angiogenic Potential

In order to calculate the effect of factors released by MSCs on the forming of HUVEC cell line in vitro tube-like structures forming, an experimental design was carried out which allowed assessing this objective under normal conditions and conditions with a reduced oxygen level; the latter condition allowed simulating an ischemia situation.

### Obtaining Supernatants in Normoxia and Hypoxia Conditions

150,000 cells of each one of the MSCs under study per bowl were planted in a plate of 6 bowls, leaving one plate for a normoxia and another for a hypoxia condition, where a humidified CO2 incubator (Thermo Scientific BBD 6220, USA), thermo-regulated at 37° C., 1% O2 was used. Cells were incubated during 48 hours in DMEM at 2% FBS. Then, supernatants were recovered and used for in vitro matrigel assay.

### In Vitro Angiogenesis Assay

In order to calculate the effect of factors released by the MSCs under normoxia and hypoxia conditions, it was necessary to use BD Matrigel matrix Growth Factor Reduced (GFR) (356230 BD, USA), a matrigel very reduced in factors that normally induce the forming of tubes. Matrigel was prepared on a 24-bowl plate, as detailed in section 7. HUVEC cells were prepared in a cellular suspension and 60,000 cells per bowl were planted on the matrigel, taking care to have a very homogeneous cell distribution on the surface. As a medium of culture, the MSCs supernatant (conditioned medium) was used in both conditions analyzed, in addition to the inclusion of a positive control using EGM and a negative control using DMEM at 2% FBS. In order to quantify the HUVEC cell line angiogenic potential in the presence of different supernatants, the same methodology described in item 8.1 was used.

### Example 10

### Presence of Soluble Factors that Induce Angiogenesis in the Supernatant of MSCs of Corion

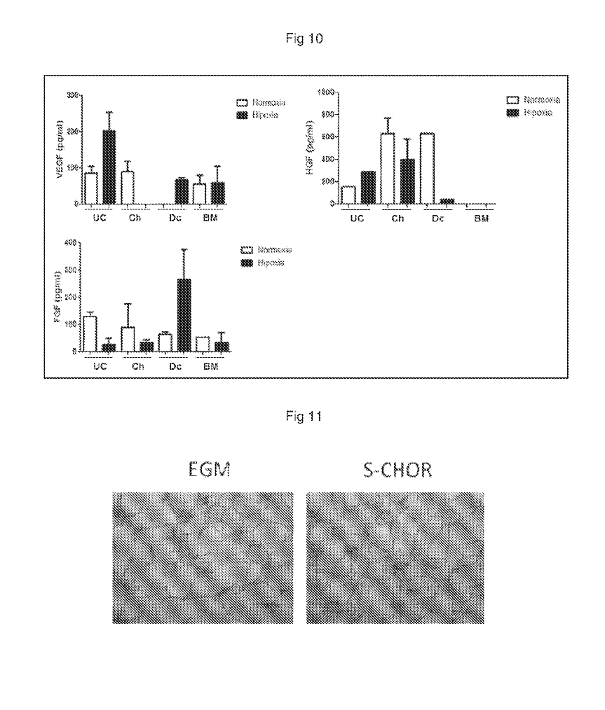

Using ELISA assays it was demonstrated that in the supernatant of MSCs Corion soluble factors that induce angiogenesis growth factor hepatocyte (HGF), the vascular endothelial growth factor (VEGF) and the fibroblast growth factor (FGF), are present as shown in FIG. 10).

The in vitro assays demonstrated the ability of the supernatant of the mesenchymal stem cells from the chorion (CHO-S-MSCs) to induce the formation of blood vessels. This ability was compared to the ability of EGM media to induce angiogenesis, which is a synthetic media containing hEGF, VEGF, and IGF-1 hFGFb, corresponding to pro-angiogenic factors. By analyzing the parameters compared CHO-S-MSCs showed significant differences with respect to the EGM to induce tube-like structures formation, suggesting a better alternative to using single factor, as it was shown in FIG. 9 C, D where the conditioned media (CHO-S-MSCs) and E is always higher than the results for EGM and also in FIG. 11, showing the differences between the in vitro tube-like structures formation for EGM defined media and S-CHOR conditioned media.

Quantification protocol for pro-angiogenic factors is standardized and their presence in the supernatant of CHO-MSCs and EGM, and further in vitro functional assays of angiogenesis are performed by comparing the angiogenic potential of MSCs-CHO supernatant with respect to the use of FGF, HGF and VEGF in the same concentration as quantified in the supernatant of CHO-MSCs. Thus it is possible to demonstrate that the use of CHO-S-MSCs as angiogenesis inducer is more efficient than the use of a synthetic composition comprising those pro-angiogenic factors commonly described.

### Example 11

### Statistics Analysis

Data represent the average value of at least three independent experiments. For statistics analysis, the t-Student test for non-parametrical data was applied in the comparison of two groups in a same source (treated v/s untreated) together with the one way ANOVA test using Bonferroni post test for non-parametrical data in the comparison of sources for a same condition. A value of p<0.05 was considered statistically significant. Results are shown with the standard diversion. The different statistics analysis was carried out with the program GraphPad Prism V.

### Example 12

### Characterization of MSCs Derived from Placenta and Bone Marrow

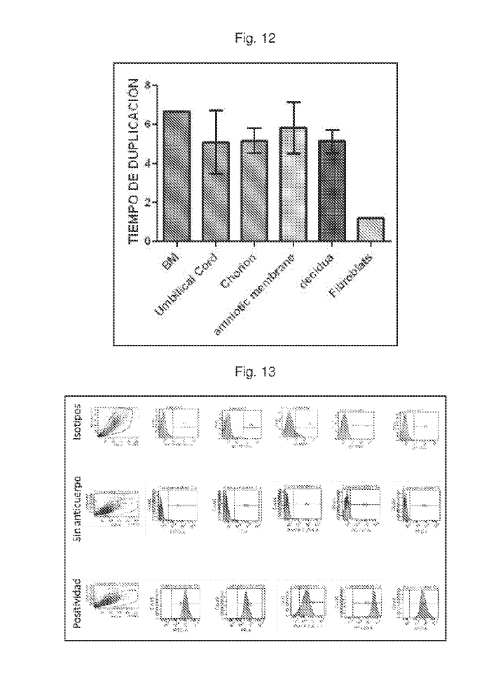

In order to establish the MSCs basic characteristics used in this research, the cell population doubling time between stages 1 and 4 was assessed. All placenta sources present a doubling time similar to bone marrow (BM) MSCs. However, data not shown in FIG. 12, and obtained at miRNA stem cells Research and Development Laboratory, and Cells for Cells, Universidad de los Andes, show that MSCs derived from the amniotic membrane, as from stage 3, suddenly decrease their doubling time. For this reason, this thesis project was carried out using MSCs derived from umbilical cord, chorion, and decidua.

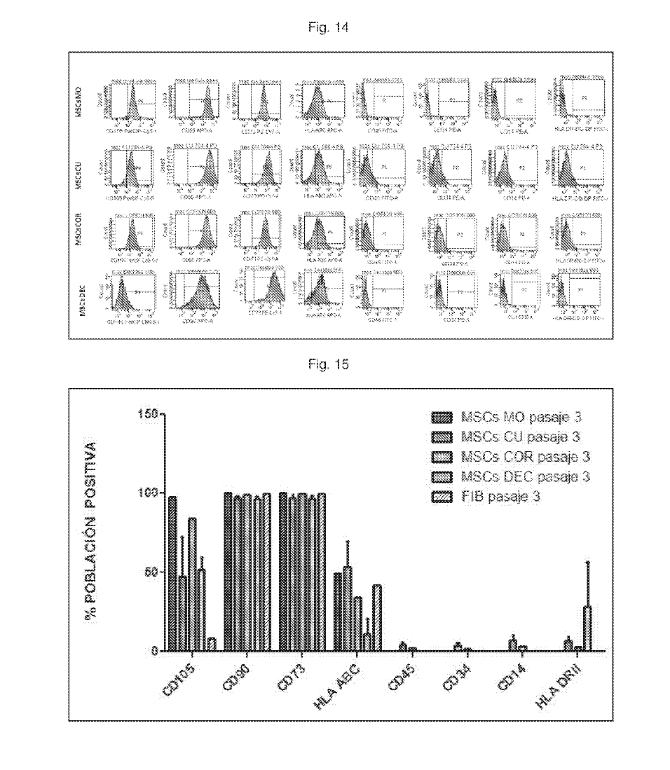

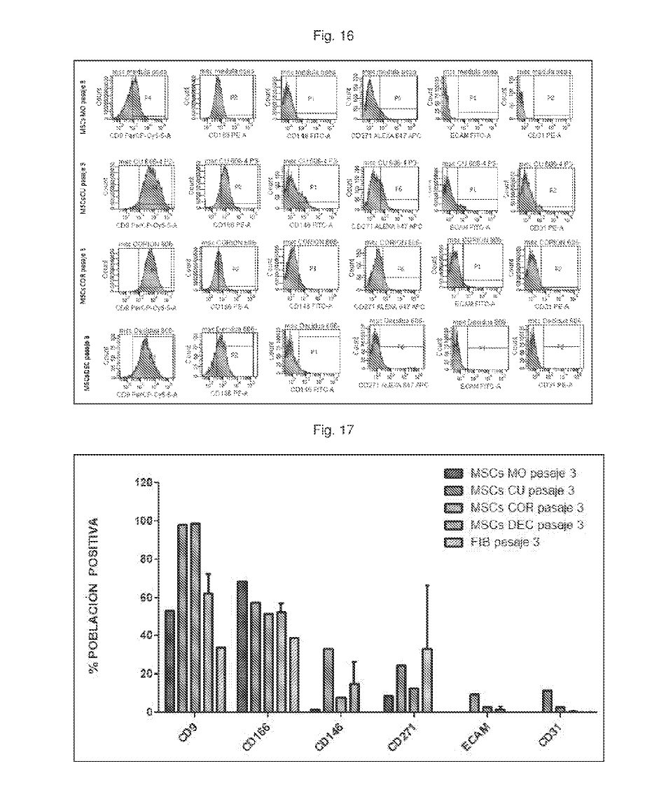

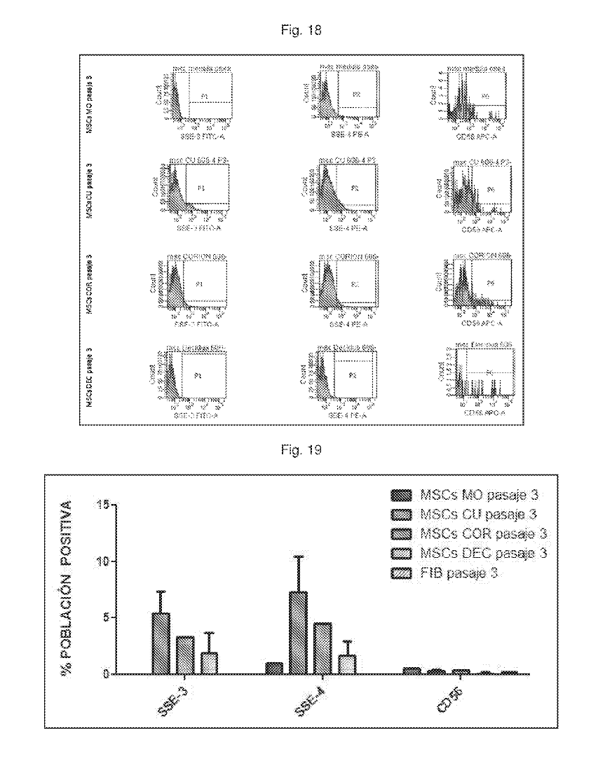

The presence of classic positive markers was also established through flow cytometry, such as CD 105, CD 90, and CD73 in MSCs derived from placenta and bone marrow. Also, MSCs were found negative for markers CD45, CD 34, CD 14, and HLA DR II, with moderate HLA ABC levels (FIGS. 13, 14, 15, 16, and 17). Furthermore, additional markers such as CD9, CD166, CD146, CD27, ECAM, and CD31 were calculated. On the other hand, the embryo markers expression such as SSE-3, SSE-4, and CD56 was assessed (FIGS. 18 and 19).

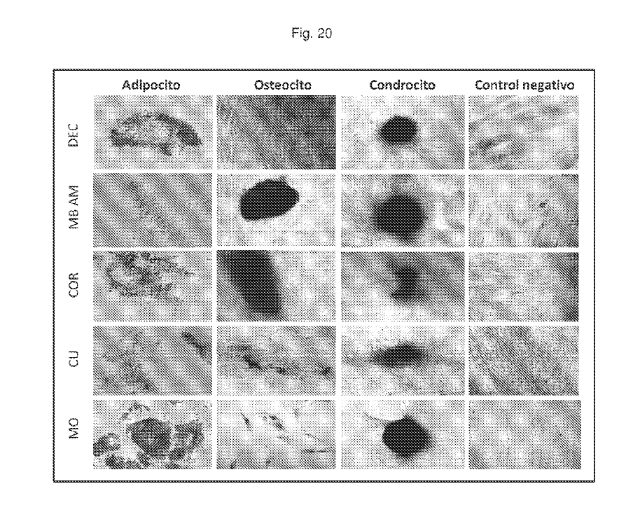

Finally, the tri-differentiation of MSCs derived from placenta and bone marrow used in this research was established. FIG. 20 shows cells differentiated to chondrocyte, adipocyte, and osteocyte, tinted with specific reagents.

What is claimed is:

1. A method of obtaining chorion-derived MSCs (mesenchymal stem cells) cells as a source of MSCs with cardio-myogenic and angiogenic potential, the method comprising:
(a) isolating chorion cells under standardized culture conditions;
(b) culturing the isolated chorion cells from step (a);
(c) inducing the cultured cells from step (b) into cardiac lineage with 5-azacytidine;
(d) evaluating cardiomiogenesis potential of the induced cells from step (c) by assessing expression of NKX2-5, GATA-4, MYH7b, GJA1 and TNNT2 genes;
(e) determining the presence of connexin 43 and cardiac troponin proteins in the induced cells from step (c);
(f) measuring the potential of angiogenesis in the induced cells from step (c) in an in vitro angiogenesis assay; and
(g) selecting the chorion-derived MSCs cells were the samples measured a cardio-myogenic and angiogenic potential higher than that shown by bone marrow MSCs.

2. The method of claim 1, wherein the step (b) comprises culturing between 2 to 6 passages.

3. The method of claim 1, wherein step (d) comprises evaluating cardiomiogenesis potential of the induced cells from step (c) by assessing with RT-qPCR the expression of NKX2-5, GATA-4, MYH7b, GJA1 and TNNT2 genes.

4. The method of claim 1, wherein step (e) comprises determining by indirect immunofluorescence.

5. The method of claim 1, wherein the in vitro angiogenesis assay in step (f) comprises evaluating the capacity of the chorion-MSCs cells to form in vitro tube-like structures.

6. Use of the chorion-derived MSCs cells obtained according to claim 1, wherein the cells are used in the clinical treatment of degenerative conditions.

7. Use of the chorion-derived MSCs cells obtained according to claim 1, wherein the cells are used in tube-like structures generation.

8. Use of the chorion-derived MSCs cells obtained according to claim 1, wherein the cells are used for the cardiac regeneration.

9. A method of obtaining a conditioned medium of chorion-derived MSCs cells obtained according to the method of claim 1, the method comprising:
culturing the chorion-derived MSCs cells under hypoxia conditions; and
collecting the supernatant of the cultured chorion-derived MSCs cells, whereby the conditioned medium is obtained.

10. The method of claim 9 wherein the cells are cultured for 48 hours.

11. Use of the conditioned medium of chorion-derived MSCs cells obtained according to claim 9, as inducer of angiogenesis.

12. Use of the conditioned medium of chorion-derived MSCs cells obtained according to claim 9, for tissue culture applications requiring pro-angiogenic factors.
